(19) United States
(12) Patent Application Publication
(10) Pub. No.: US 2006/0116674 A1
(43) Pub. Date: Jun. 1, 2006

(54) Method of regenerating the recticular architecture of the dermis

(75) Inventors: Nigel M. Goble, Hungerford (GB); Colin C.O. Goble, Henley-on-Thames (GB); Keith Penny, Monmouth (GB)

Correspondence Address: OLIFF & BERRIDGE, PLC, P.O. BOX 19928, ALEXANDRIA, VA, 22320, US

(73) Assignee: RHYTEC LIMITED, Hungerford (GB)

(21) Appl. No.: 11/281,594

(22) Filed: Nov. 18, 2005

Related U.S. Application Data
(63) Continuation-in-part of application No. 10/792,765, filed on Mar. 5, 2004.
Application No. 10/792,765 is a continuation-in-part of application No. 09/789,550, filed on Feb. 22, 2001, now Pat. No. 6,723,091.
(60) Provisional application No. 60/183,785, filed on Feb. 22, 2000.
(60) Provisional application No. 60/653,498, filed on Feb. 17, 2005.

(30) Foreign Application Priority Data
Feb. 17, 2005 (GB) 0503330.3

(51) Int. Cl. A61B 18/18

(52) U.S. Cl. 606/41; 606/34

(57) ABSTRACT

A cosmetic method of regenerating the reticular architecture of tissue is disclosed. The method uses a source of thermal energy with a low thermal time constant, and comprises the step of operating the thermal energy source to form first and second adjacent regions of thermally-modified tissue in the region of the DE Junction. The first region overlies the second region and is thermally modified to a greater extent than the second region.

This application claims priority of U.S. Provisional Patent Application No. 60/653,498, filed Feb. 17, 2005. This application is a Continuation-in-Part of U.S. patent application Ser. No. 10/792,765, filed Mar. 5, 2004 that is a Continuation-in-Part Application of U.S. patent application Ser. No. 09/789,500, filed Feb. 22, 2001, that in turn claims the benefit of priority of U.S. Provisional Patent Application No. 60/183,785, filed Feb. 22, 2000. The complete disclosures of U.S. Provisional Patent Application No. 60/653,498, U.S. patent application Ser. No. 10/792,765, U.S. patent application Ser. No. 09/789,500, and U.S. Provisional Patent Application No. 60/183,785, including the specifications, drawings, and claims are incorporated herein by reference in their entirety.

### BACKGROUND OF THE INVENTION

1. Field of the Invention

This invention relates to a method of regenerating the reticular architecture of the dermis.

Human skin has two principal layers: the epidermis, which is the outer layer and typically has a thickness of around 120μ in the region of the face, and the dermis which is typically 20-30 times thicker than the epidermis, and contains hair follicles, sebaceous glands, nerve endings and fine blood capillaries. By volume the dermis is made up predominantly of the protein collagen.

Ageing and exposure to ultraviolet (UV) light result in changes to the structure of the skin, these changes including a loss of elasticity, sagging, wrinkling and a pallor or yellowing of the skin consistent with reduced vascularity.

On histological examination, these changes can be seen as a loss of the undulations or rete pegs at the junction of the epidermis and dermis (the DE Junction), reducing the surface area and vascularity of the basement membrane from which the epidermis is generated. This is accompanied by a lack of polarisation and flattening of the cells at the basement membrane as they become less active. Changes due to UV exposure, often termed photodamage, result in the formation of a layer of collagen laid down parallel to the DE Junction called the Grenz zone. In normal skin, the layer below the DE Junction is often termed the papillary dermis, and below that the reticular dermis. Both areas are nonetheless rich in the reticular elements of the dermis, including elastin, collagen and glycosaminoglycans (GAGS). These are responsible for the retention of fluids in the healthy dermis and for its elastic and structural properties. Typically, the proportion of elastin in the papillary dermis is higher than that of collagen, with the GAGS molecular matrices sitting between the two filamentous structures. Normally, the elastin is finer than the collagen; and, at the DE Junction, even finer fibres of fibrillin are often observed anchoring the basal layer. With the appearance of the Grenz zone, these fibrillin fibres are seen to be absent, and below the zone the elastin will be seen to be abnormal, clumped and thickened. This appearance is termed solar elastosis, and typifies the changes of photodamage. The process by which this happens is complicated, but in essence relates to a derangement of fibroblasts and enzyme systems by UV exposure that otherwise are involved in recycling ageing or damaged reticular structures. Along with elastosis, the proportions of GAGS and the various types of collagen fibres also become abnormally altered.

A common aim of many cosmetic procedures is to improve the appearance of a patient's skin. For example, a desirable clinical effect in the field of cosmetic procedures is to provide an improvement in the texture of ageing skin, and to give it a more youthful appearance. These effects can be achieved by the removal of a part or all of the epidermis, and on occasions part of the dermis, causing the growth of a new epidermis having the desired properties.

One known technique for achieving skin resurfacing includes the mechanical removal of tissue by means of an abrasive wheel, for example. Another technique is known as a chemical peel, and involves the application of a corrosive chemical to the surface of the epidermis, to remove epidermal, and possible dermal skin cells. Yet a further technique is laser resurfacing of the skin. Lasers are used to deliver a controlled amount of energy to the epidermis. This energy is absorbed by the epidermis causing necrosis of epidermal cells. Necrosis can occur either as a result of the energy absorption causing the temperature of the water in the cells to increase to a level at which the cells die, or alternatively, depending upon the frequency of the laser light employed, the energy may be absorbed by molecules within the cells of the epidermis in a manner which results in their dissociation. This molecular dissociation kills the cells, and as a side effect also gives rise to an increase in temperature of the skin.

All these methods are referred to as non-surgical techniques, as they are not associated with an incision or surgical manipulation of the tissue as occurs in, for example, a surgical face-lift where an incision is made through the skin, redundant skin is removed and, when the incision is closed, the skin is pulled taut. The effects of these non-surgical methods rely on the healing response of the skin to the superficial injury whether this be heat (laser), mechanical (microdermabrasion), or chemical (peel) so that they must not go “through the skin” or a scar would result as occurs with a surgical incision. The disadvantage of each of these methods is that the surface of the skin is effectively removed at the time of the procedure, and that the depth of effect is dependent on the depth of the skin removed at that time. There is little or no modification of tissues beneath the point of removal, so that it is the formation of scar tissue at the level of removal that provides the result.

Another known non-surgical skin treatment is known as thermage. Thermage, which was approved by the FDA in 2002 for the area around the eyes, is now used to treat whole faces. It uses a radio frequency device to heat the lower layers of the skin, while protecting the outer layers with a cooling spray. The result is a tightening of the facial layers that is not quite a facelift, but is as close as you can get without surgery. The disadvantage of this treatment is that it is painful, because the frequency is conducted along nerve endings.

Plasma Skin Regeneration (PSR) is a non-surgical technique employing an invention disclosed in U.S. patent application Ser. No. 10/792,765, filed 5 Mar. 2004, the disclosure of which (including the specification, drawings and claims) is incorporated by reference in its entirety. The method of treating the skin using PSR involves exposing the skin to millisecond pulses of nitrogen or other diatomic gas that has been ionised using ultra-high frequency radiofrequency energy. The ionised gas stores energy that is given up to the skin as thermal energy producing a heating of both the epidermis and deeper dermis of the skin. The depth of the effect is a function of the power setting and the moisture content of the skin, provided the distance and angle of the plasma pulse remains constant with respect to the skin surface.

The energy locked up in the nitrogen gas takes the form of ionisation, splitting of the nitrogen molecules and oscillatory motions of the molecules. On impact with the skin, this energy is given up directly to the fluid content of the skin to vaporise at least part of the skin. As heat is given up to the skin as a whole, variations in water content will modify its bulk thermal characteristics. No intermediary is involved, as occurs with lasers that rely on a target chromophore for conversion of light energy to thermal energy. The effect is more unform and less disruptive as a result. Consistent with this, the treatment of photodamage using lasers often involves more than one pass over the surface, with the treated skin being wiped away between passes. The wiping is necessary, not only to increase the depth of penetration, but also to refresh the chromophore.

2. Cross-Reference to Related Patents and Patent Applications

A tissue-treatment system is disclosed in related U.S. Pat. No. 6,629,974, filed Feb. 13, 2002 and U.S. Pat. No. 6,723,091, filed Feb. 22, 2001. The complete disclosures of U.S. Pat. No. 6,629,974 and U.S. Pat. No. 6,723,091, including the specifications, drawings, and claims are incorporated herein by reference in their entirety.

### SUMMARY OF THE INVENTION

The present invention provides a cosmetic method of regenerating the reticular architecture of tissue using a source of thermal energy with a low thermal time constant, the method comprising the step of operating the thermal energy source to form first and second adjacent regions of thermally-modified tissue in the region of the DE Junction, said first region overlying said second region and being thermally modified to a greater extent than said second region.

The invention also provides a cosmetic method of regenerating the reticular architecture of the dermis using a source of thermal energy with a low thermal time constant, the method comprising the step of operating the thermal energy source and directing it at the surface of the skin to form first and second adjacent regions of thermally-modified tissue in the region of the epidermis and dermis of the skin, said first region overlying said second region and being thermally modified to an extent that it separates from said second region some days after the delivery of the thermal energy, and the depth of said separation being dependent on the amount of energy delivered and the thermal capacity of the skin.

In a preferred embodiment, the thermal energy source is operated for a singe pass over the skin surface, the thermal energy source being arranged to have an energy setting dependent on the desired depth of effect. Alternatively, the thermal energy source is operated over at least two passes over the skin surface, the energy levels of the passes being chosen dependent on the desired depth of effect.

In either case, the energy setting of the thermal energy source may be such as to create vacuolation on the first pass. In the latter case, the energy setting of the thermal energy source may be such as not to create vacuolation on the first pass, thereby enabling a second pass without removing the treated skin.

Preferably, the energy setting of the thermal energy source is such as to preserve the integrity of the epidermis as a biological dressing.

In a preferred embodiment, the thermal energy source is operated so that a line of cleavage occurs within the skin 2 to 5 days following treatment, the line of cleavage occurring between said first and second regions. In one particular case, the operation of the thermal energy source may be such as to form a line of cleavage from 2 to 3 cells deep in the stratum corneum of the superficial epidermis and the upper dermis.

Advantageously, the operation of the thermal energy source is such that the tissue in the first region is sloughed tissue. In this case, the sloughed tissue is removed once a new epidermis has been substantially generated in the region of the line of cleavage.

Preferably, the tissue below the line of cleavage in said second region includes the lower epidermis, the basal membrane and the DE Junction. More preferably, at least the thermally-modified basal membrane and the DE Junction are regenerated.

In one particular case, the line of cleavage forms below areas of solar elastosis, such that the solar elastosis and deranged fibroblasts are sloughed.

Preferably, the operation of the thermal energy source is such as to denature dermal collagen in the second region.

In a preferred embodiment, the tissue in said second region undergoes a regenerative process following regeneration of the epidermis.

In this case, the reticular architecture of the dermis is regenerated in whole, or in part, by fibroblasts less exposed to the effects of UV radiation.

The collagen architecture and/or elastin architecture and/or the GAGS of the dermis is regenerated in whole, or in part, by fibroblasts less exposed to the effects of UV radiation.

Preferably, the healing process is such that risk of scarring and hypo pigmentation is substantially eliminated.

Advantageously, a progressive improvement in skin changes associated with ageing and photodamage occur over a period of between 6 and 12 months following treatment.

In a preferred embodiment, the source of thermal energy is an instrument having an electrode connected to a power output device, and wherein the power output device is operated to create an electric field in the region of the electrode; a flow of gas is directed through the electric field to generate, by virtue of the interaction of the electric field with the gas, a plasma; the plasma is directed onto the tissue for a predetermined period of time; and the power transferred into the plasma from the electric field is controlled so as to desiccate at least a portion of the dermis with vapour pockets formed in dermis cells.

Preferably, the power output device is operated to deliver discrete pulses of heat of millisecond duration.

Advantageously, the pulses have a duration in the range of from about 0.5 to about 100 milliseconds, and preferably a duration in the range of from about 4.5 to about 15.4 milliseconds.

Preferably, the flow of gas is directed through a nozzle of the instrument.

Conveniently, the power output device is operated to deliver energy in the range of from about 1 Joule to about 4 Joules for an instrument having a first predetermined nozzle diameter, and to deliver energy in the range of from less than 0.5 Joules to about 2 Joules for an instrument having a second predetermined diameter that is less than the first predetermined diameter.

Preferably, the first predetermined diameter is substantially 5 mm, and the second predetermined diameter is substantially 1.5 mm.

In a preferred embodiment, the thermal energy source is operated to direct a jet of fluid having stored heat energy at the skin surface. Advantageously, the jet of fluid is a jet of an ionised diatomic gas.

The method provided by the invention is not a therapeutic method, it is preferably a cosmetic method that may be carried out to improve the appearance of the skin.

### BRIEF DESCRIPTION OF THE DRAWINGS

Embodiments of the invention will now be described, by way of example and with reference to the accompanying drawings, in which:

FIG. 1 is a diagrammatic view of a tissue treatment system in accordance with the invention;

FIG. 2 is a longitudinal cross-section of a tissue treatment instrument forming part of the system of FIG. 1;

FIG. 3 is a block diagram of a radio frequency generator for use in the system of FIG. 1;

FIG. 4 shows the regeneration of the reticular architecture of the dermis when using the system of FIGS. 1 to 3 for different pulse widths and energy ratings; and

FIGS. 5 to 7 show the process of reticular regeneration at the day of treatment, at four days after treatment, and at ten days after treatment respectively.

### DETAILED DESCRIPTION OF THE PREFERRED EMBODIMENTS

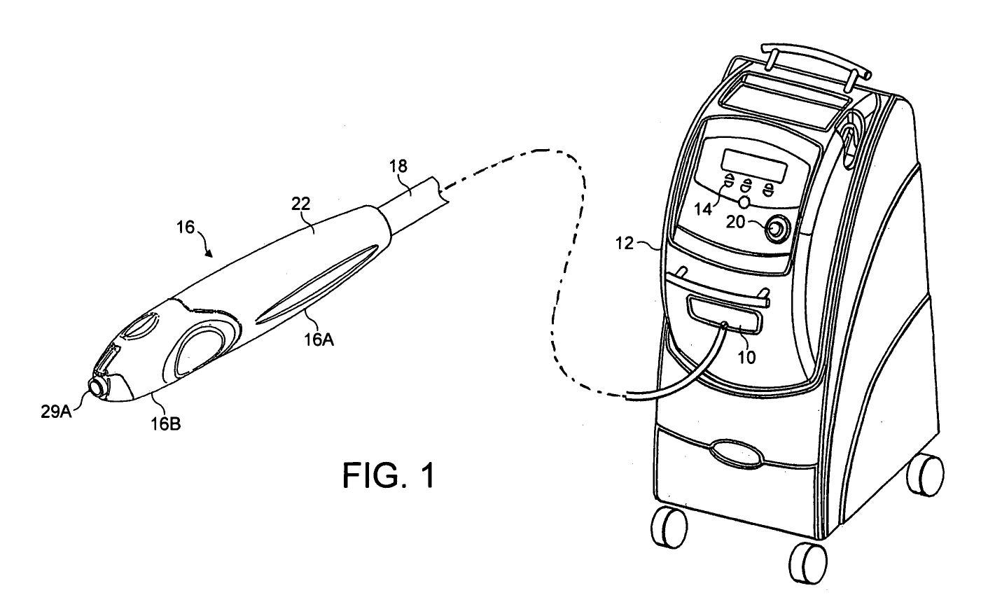

Referring to FIG. 1, a tissue treatment system in accordance with the invention has a treatment power source in the form of a radio frequency (r.f.) generator 10 mounted in a floor-standing generator housing 12 and having a user interface 14 for setting the generator to different energy level settings. A handheld tissue treatment instrument 16 is connected to the generator by means of a cord 18. The instrument 16 comprises a handpiece having a re-usable handpiece body 16A and a disposable nose assembly 16B.

The generator housing 12 has an instrument holder 20 for storing the instrument when not in use.

Within the cord 18 there is a coaxial cable for conveying r.f. energy from the generator 10 to the instrument 16, and a gas supply pipe for supplying nitrogen gas from a gas reservoir or source (not shown) inside the generator housing 12. The cord also contains an optical fibre line for transmitting visible light to the instrument from a light source in the generator housing. At its distal end, the cord 18 passes into the casing 22 of the handpiece body 16A

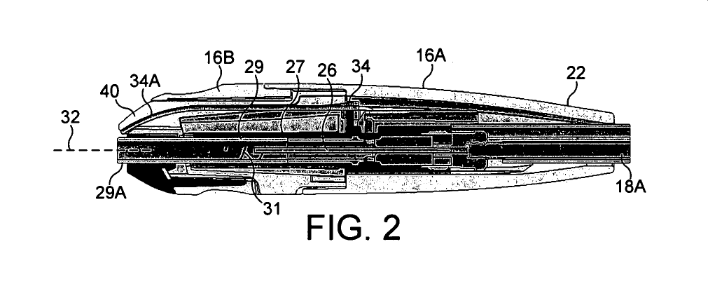

In the re-usable handpiece body 16A, the coaxial cable 18A is connected to inner and outer electrodes 26 and 27, as shown in FIG. 2. The inner electrode 26 extends longitudinally within the outer electrode 27. Between them is a heat-resistant tube 29 (preferably made of quartz) housed in the disposable instrument nose assembly 16B. When the nose assembly 16B is secured to the handpiece body 16A, the interior of the tube 29 is in communication with the gas supply pipe interior, the nose assembly 16B being received within the body 16A such that the inner electrode 26 extends axially into the tube 29 and the outer electrode 27 extends around the outside of the tube 29.

A resonator in the form of a helically wound tungsten coil 31 is located within the quartz tube 29, the coil being positioned such that, when the disposable nose assembly 16B is secured in position on the handpiece body 16A, the proximal end of the coil is adjacent the distal end of the inner electrode 26. The coil is wound such that it is adjacent and in intimate contact with the inner surface of the quartz tube 29.

In use of the instrument, nitrogen gas is fed by a supply pipe to the interior of the tube 29 where it reaches a location adjacent the distal end of the inner electrode 26. When an r.f. voltage is supplied via the coaxial cable to the electrodes 26 and 27, an intense r.f. electric field is created inside the tube 29 in the region of the distal end of the inner electrode. The field strength is aided by the helical coil 31 which is resonant at the operating frequency of the generator and, in this way, conversion of the nitrogen gas into a plasma is promoted, the plasma exiting as a jet at a nozzle 29A of the quartz tube 29. The plasma jet, centred on a treatment beam axis 32 (this axis being the axis of the tube 29), is directed onto tissue to be treated, the nozzle 29A typically being held a few millimetres from the surface of the tissue.

The handpiece 16 also contains an optical fibre light guide 34 which extends through the core 18 into the handpiece where its distal end portion 34A is bent inwardly towards the treatment axis defined by the quartz tube 29 to terminate at a distal end which defines an exit aperture adjacent the nozzle 29A. The inclination of the fibre guide at this point defines a projection axis for projecting a target marker onto the tissue surface, as will be described in more detail below.

Following repeated use of the instrument, the quartz tube 29 and its resonant coil 31 require replacement. The disposable nose assembly 16B containing these elements is easily attached and detached from the reusable part 16A of the instrument, the interface between the two components 16A, 16B of the instrument providing accurate location of the quartz tube 29 and the coil 31 with respect to the electrodes 26, 27.

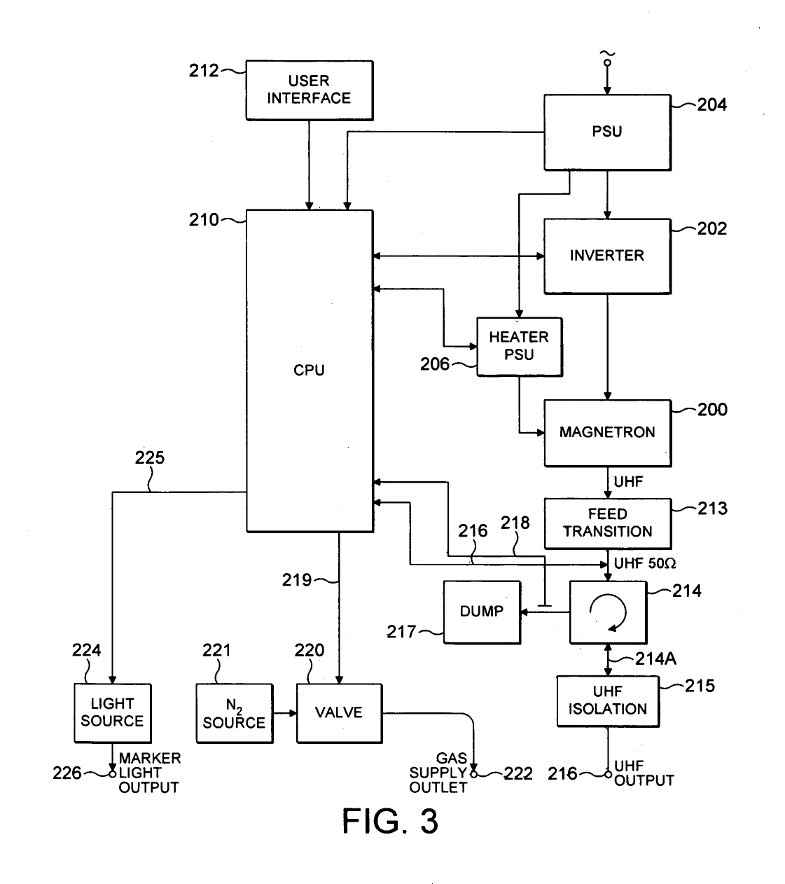

Referring to FIG. 3, r.f energy is generated in a magnetron 200. Power for the magnetron 200 is supplied in two ways, firstly as a high DC voltage for the cathode, generated by an inverter 202 supplied from a power supply unit 204 and, secondly, as a filament supply for the cathode heater from a heater power supply unit 206. Both the high voltage supply represented by the inverter 202 and the filament supply 206 are coupled to a CPU controller 210 for controlling the power output of the magnetron. A user interface 212 is coupled to the controller 210 for the purpose of setting the power output mode, amongst other functions.

The magnetron 200 operates in the high UHF band, typically at 2.475 GHz, producing an output on an output line which feeds a feed transition stage 213 for converting the waveguide magnetron output to a coaxial 50 ohms feeder, low frequency AC isolation also being provided by this stage. Thereafter, a circulator 214 provides a constant 50 ohms load impedance for the output of the feed transition stage 213. Apart from a first port coupled to the transition stage 213, the circulator 214 has a second port 214A coupled to a UHF isolation stage 215 and hence to the output terminal 216 of the generator for delivering RF power to the handheld instrument 16 (FIG. 1). Reflected power is fed from the circulator 214 to a resistive power dump 215. Forward and reflected power sensing connections 216 and 218 provide sensing signals for the controller 210.

The controller 210 also applies via line 219 a control signal for opening and closing a gas supply valve 220 so that nitrogen gas is supplied from the source 221 to a gas supply outlet 222 from where it is fed through the gas supply pipe in the cord 18 to the instrument 16 (FIG. 1), when required. A light source 224, forming part of the above-mentioned optical target marker projector, is connected to the controller 210 by a control line 225 and produces a target marker light beam at an optical marker light output 226.

The controller 210 is programmed to pulse the magnetron 200 so that, when the user presses a footswitch (not shown in the drawings), r.f. energy is delivered as a pulsed waveform to the UHF output 216, typically at a pulse repetition rate of about 4 Hz. The controller 210 also operates the valve 220 so that nitrogen gas is supplied to the handheld instrument simultaneously with the supply of r.f. energy. The light source 224 can be actuated independently of r.f. energy and nitrogen gas supply. Further details of the modes of delivery of r.f. energy are set out in U.S. Pat. No. 6,723,091, filed on 22 Feb. 2001, the disclosure of which (including the specification, the drawings and the claims) is incorporated herein by reference in its entirety.

In use, the instrument 16 is passed over the surface of tissue to be cosmetically treated, with the nozzle 29a typically being held a few millimetres from the surface of the tissue. The pulse duration and energy levels are chosen so as to form first and second adjacent regions of thermally-modified tissue in the region of the DE Junction. The first, upper region is termed a zone of thermal damage, having a thermal modification which is greater than that of the second, lower region. The thermally damaged zone is thermally modified to an extent that it separates from the second region some days after the delivery of the thermal energy. Following separation of the first damaged region, the epidermis and the upper region of the dermis regenerate naturally.

A benefit of using a diatomic plasma is that it is able to deliver a relatively large amount of energy which causes heating in a short period of time. This enables delivery in discreet pulses of millisecond duration, and is in contrast to heat conduction from a merely hot gas. In the preferred embodiment, energy from 1 Joule to 4 Joules is delivered in a period of 4.5 to 15.4 milliseconds respectively for a nozzle with an exit diameter of 5 millimetres, and delivers from less than 0.5 Joules up to 2 Joules in the same period for a nozzle with an exit diameter of less than 1.5 millimetres. Experiments have shown that useful clinical effects are achieved with yet longer pulses extending to 50 milliseconds, and further analysis shows extension up to 100 milliseconds or more will provide useful effects. In addition, the pulse width may be shortened to deliver the same, or otherwise similar, useful heating energy. Plasma pulses as short as 0.5 milliseconds have been produced with the system described above.

Another benefit is that oxygen is purged from the skin surface by the plasma and flow of inert gas that follows immediately following a plasma pulse. As a result, the oxidative carbonisation that often occurs at the skin surface on application of thermal energy is avoided, leaving a desiccated intact epithelium with minor structural alteration.

This minor structural alteration is nonetheless important in providing yet another benefit of the invention, as it changes the thermal characteristics of the epidermis at higher energy settings. Following a single pass of plasma over the skin surface at an energy setting greater than 2 Joules, the epidermal cells at the basal membrane are heated to a degree that produces vacuolation of the cellular contents. This produces a natural insulator limiting the absorption and depth of penetration of energy from subsequent passes. This is a beneficial safety feature that avoids the risk of excessive damage by inadvertent application of multiple passes to the same site on the skin surface.

Alternatively, when using energy pulses at or below 2 Joules, then the vacuolation is not observed, and the treated skin is still capable of absorbing the thermal energy of a second pass, by changing the energy in the second pass using either a narrow nozzle to focus the plasma or a higher energy setting will have an additive effect. The benefit of using a narrow nozzle embodiment is that the focused energy can be directed onto specific areas of the skin surface such as deeper wrinkles.

For example, if the skin is subjected to two passes of 4 Joules, then the depth of thermal effect is only 10-20% greater than with a single pass of 4 Joules. Alternatively, if the skin is first treated with 2 Joules, then with a second pass of 4 Joules then the effect will be consistent with a single pass with 6 Joules. Part of this benefit also relates to the water content of the skin, particularly the upper layers of the epidermis following pre-treatment with a topical anaesthetic.

Through experimentation with the invention, it has become clear that the depth of effect changes by up to 50% depending on the hydration of the upper layers of the epidermis following application of a topical anaesthetic. Topical anaesthetics include a hydrating component, as they rely on hydration of the superficial epidermis for the penetration of the anaesthetic agent through the skin. This changes the absorption of pure thermal energy, whereby a larger proportion of the energy is dissipated in the superficial epidermis, reducing the depth of penetration into the dermis. If no anaesthesia or tumescent subcuticular anaesthesia is employed, then the depth of dermal penetration for a given energy setting can be doubled. A pre-treatment with 2 Joules produces sufficient desiccation of the superficial epidermis, following use of topical anaesthesia, that an equivalent depth of effect can be produced with the second pass to that achieved with no anaesthesia or tumescent subcuticular anaesthesia.

The reason for using a diatomic plasma which delivers a relatively large amount of energy in a short period of time is that the irreversible clinical effects (the thermal modification and thermal damage of the tissue) occur over tissue depths that result in the desired clinical effects, whilst avoiding any undesired clinical effects. If the heating energy is delivered over too long a time, the effects of convection from the skins surface and conduction into the underlying tissue will be such that no significant temperature rise results. On the other hand, if the time is too short, then irreversible effects (such as water vaporising) at or near the skins surface will carry away otherwise useful heating energy.

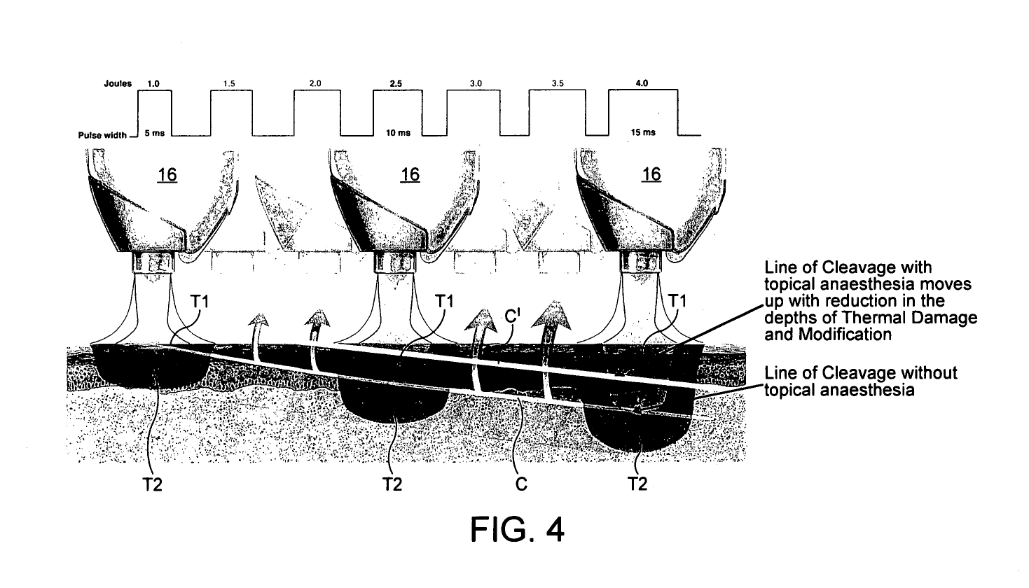

FIG. 4 shows the regeneration of the reticular architecture of the dermis for different pulse widths and energy ratings, and illustrates the use of a thermal source with a low thermal time constant. Thus, for an energy setting of 1 Joule, a pulse width of 5 milliseconds is appropriate, for an energy setting of 2.5 Joules, a pulse width of 10 milliseconds is appropriate, and for an energy setting of 4 Joules, a pulse width of 15 milliseconds is appropriate. FIG. 4 also shows the two regions of thermal modification T1 and T2, T1 being the upper region of thermal damage, and T2 being the lower region of thermal modification. FIG. 4 also shows the line of cleavage C which develops between these two regions between two and five days after treatment. As is apparent, the depth of effect increases as the energy level and pulse width used for the treatment increases. The dermatologist carrying out the procedure will, therefore, choose the appropriate energy level and pulse width depending on the depth of effect required.

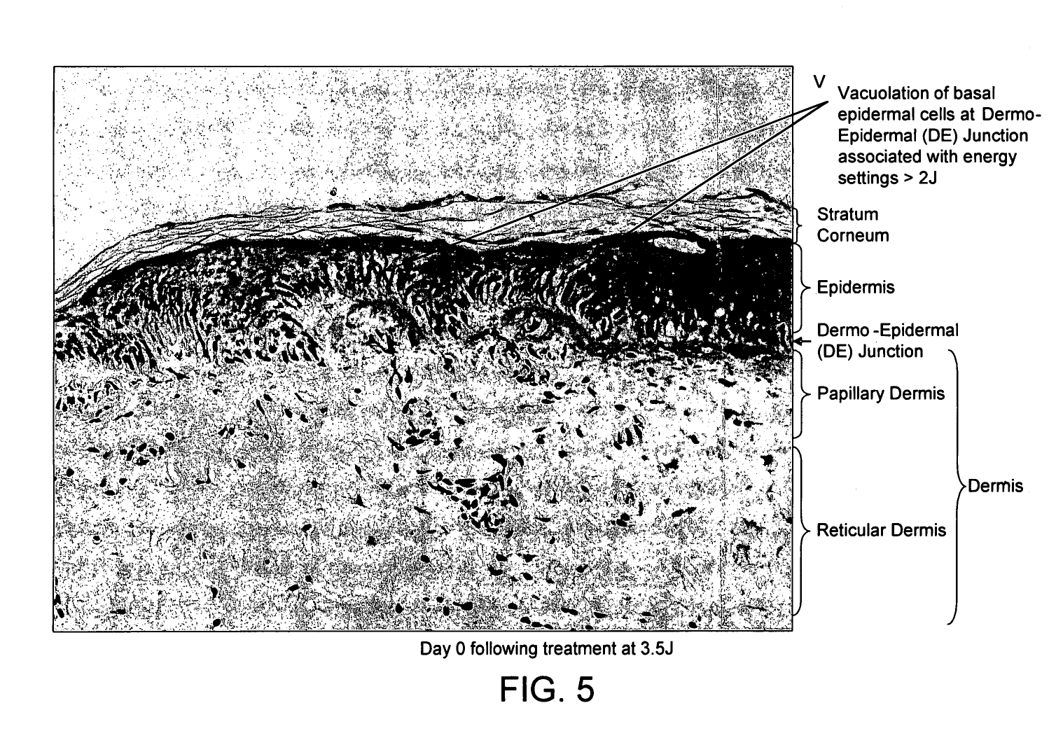

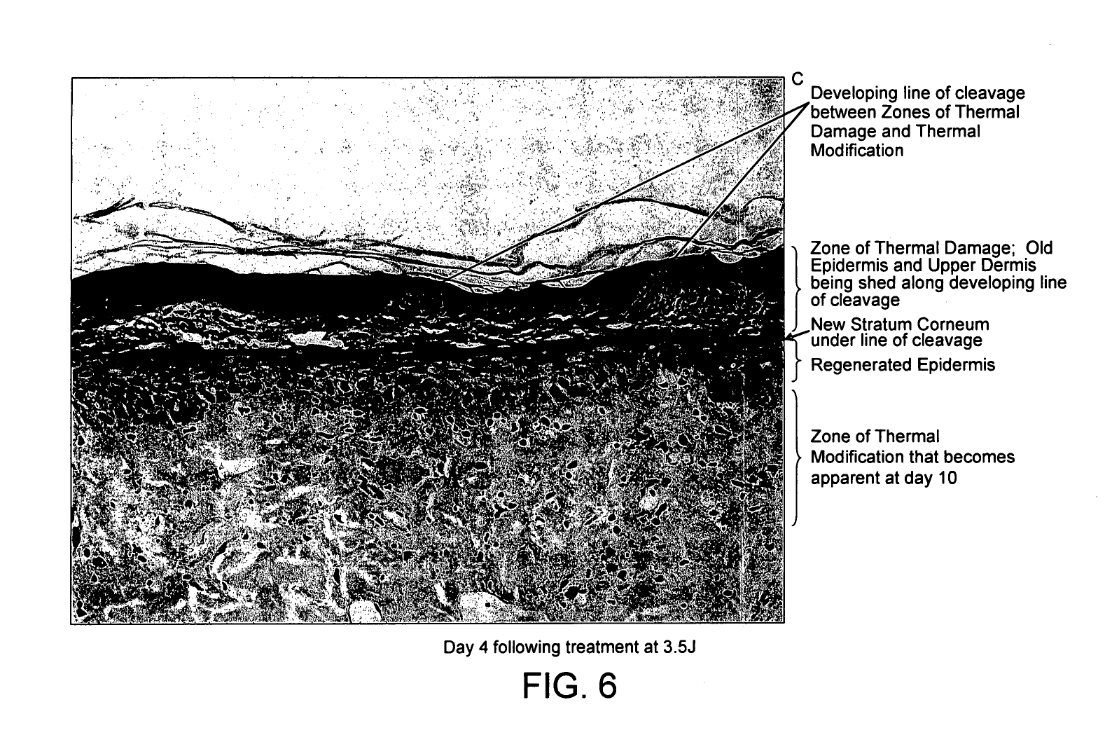

As mentioned above, the use of a topical anaesthetic modifies the effect of the treatment. Thus, as shown in FIG. 4 the line of cleavage C is for treatment without a topical anaesthetic, the equivalent line of cleavage (C1) being higher, owing to a reduction in the depth of thermal damage and modification which results from pre-treatment with a topical anaesthetic. FIGS. 5 to 7 show a typical treatment, and the progress of regeneration of the reticular architecture after the treatment. Thus, FIG. 5 shows the effect of treatment at 3.5 Joules and a pulse width of 13.6 milliseconds immediately following treatment. The Figure shows the dermis (including the reticular dermis and the papillary dermis), the DE Junction, the epidermis and the stratum corneum. Vacuolation of basal epidermal cells at the DE Junction is clearly visible, as indicated by the reference V. FIG. 6 shows the position at day four following treatment at 3.5 Joules, and shows a developing line of cleavage C between the regions T1 and T2 of thermal damage and thermal modification. The region T1 of thermal damage is the old epidermis and the upper dermis, which is in the process of being shed along the developing line of cleavage C. Underneath the line of cleavage C a new stratum corneum and a regenerated epidermis are being developed naturally. FIG. 6 also shows the zone where thermal modification will later become apparent.

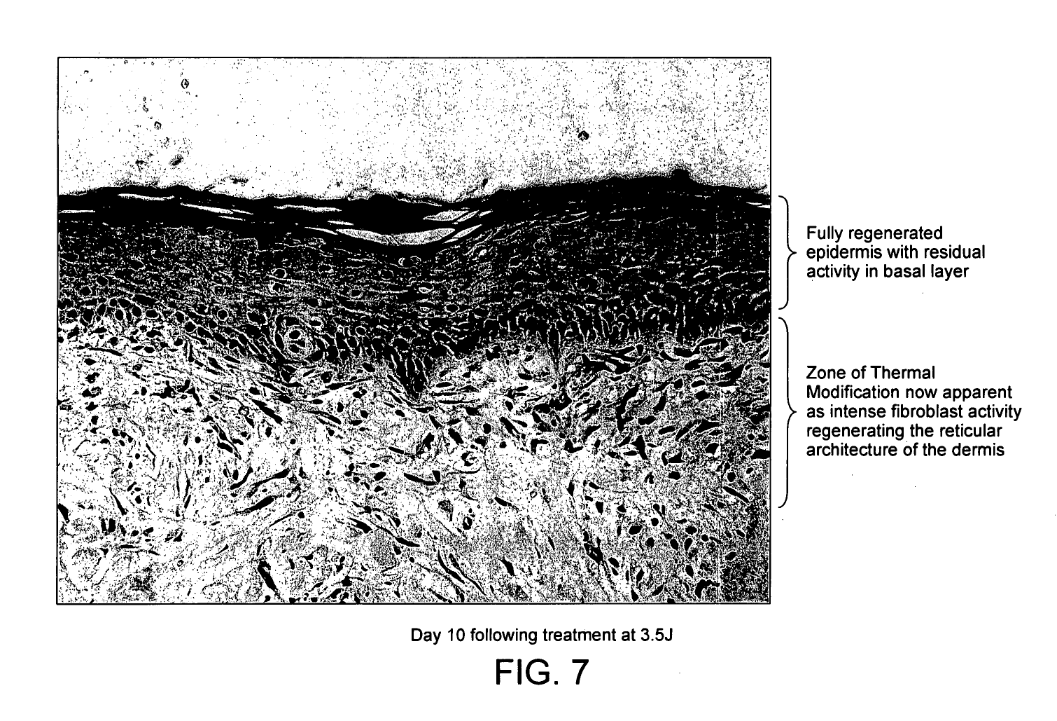

FIG. 7 shows the position at day ten following treatment at 3.5 Joules. Here, the epidermis has been fully regenerated with residual activity in the basal layer, and the zone of thermal modification is now apparent, as intense fibroblast activity regenerates the reticular architecture of the dermis.

To one skilled in the art, it is apparent that the above effects, and method described below, can be achieved using the delivery of heating energy to the skin that has the characteristics of a low thermal time constant, delivery in very short duration pulses (typically 0.5 to 10 milliseconds), and that does not rely on an intermediary conversion from one energy form to another, such as a chromophore in laser energy and tissue resistivity in radio frequency energy.

It will also be apparent that mechanisms other than a plasma device may be used to deliver the heating energy. In principle, any heating source, for example a material such as a hot gas, a condensing gas such as steam, a hot liquid or a hot solid that can produce in the tissue similar changes of temperature over time and tissue depth as produced by the plasma device will produce similar clinical effects. It would also be possible to use electromagnetic radiation (including light) of appropriate frequency. A further possibility would be to heat using a local exothermic chemical reaction.

In practice, it is necessary that such mechanisms are able to deliver a similar amount of energy per unit area in a similar amount of time as that described for a plasma, to achieve the required temperature. It is necessary that such a material must have the attributes of a small thermal time constant, so that the required energy can be delivered in the required amount of time. The thermal time constant is related to a particular object rather than a particular material, but is dependent also on the thermal characteristics of the material. For example, a small hot object will rapidly cool when in contact with a cooler object, yet a larger body at the same initial temperature will cool more slowly and deliver more heating energy, even though both may have the same contact area, and be made of the same material.

The thermal time constant of an object is a measure of the time taken for the temperature of the body to fall to 0.368 of its original temperature due to heat loss to a cooler body. It is apparent, therefore, that the thermal time constant of a hot object used to deliver a similar heating effect should be of the same order as the plasma pulse width.

What is claimed is:

1. A cosmetic method of regenerating the reticular architecture of tissue, comprising:
operating a thermal energy source with a low thermal time constant to form first and second regions of thermally-modified tissue in a region of the epidermis and the dermis of a patient's skin, the first region adjacent to the second region, the first region overlying the second region, and the first region being thermally modified to a greater extent than the second region.

2. The method of claim 1, wherein operating the thermal energy source comprises:
directing the thermal energy source at a surface of the patient's skin to form the first and second regions of thermally-modified tissue, the first region being thermally modified to the extent that the first region separates from the second region a predetermined time period after directing the thermal energy at the surface of the patient's skin, and a depth of the separation being dependent on an amount of energy directed at the surface of the patient's skin and a thermal capacity of the patient's skin.

3. The method of claim 1, wherein operating the thermal energy source comprises:
operating the thermal energy source with the low thermal time constant to form the first and the second regions of thermally-modified tissue in a region of the DE Junction.

4. The method of claim 1, wherein operating the thermal energy source comprises:
operating the thermal energy source for a single pass over the patient's skin surface; and
setting an energy setting of the thermal energy source dependent on a desired depth of effect.

5. The method of claim 1, wherein operating the thermal energy source comprises:
operating the thermal energy source for at least two passes over the patient's skin surface; and
choosing an energy level for each of the at last two passes dependent on the desired depth of the effect.

6. The method of claim 5, wherein setting the energy setting of the thermal energy source comprises:
setting the energy setting of the thermal energy source in order to create vacuolation on a first pass.

7. The method of claim 5, wherein:
setting the energy setting of the thermal energy source comprises setting the energy setting of the thermal energy source in order to avoid vacuolation on a first pass; and
operating the thermal energy source for at least two passes over the patient's skin surface comprises operating the thermal energy source for a second pass without removing treated skin.

8. The method of claim 1, further comprising:
setting an energy setting of the thermal energy source in order to preserve the integrity of the epidermis as a biological dressing.

9. The method of claim 1, wherein operating the thermal energy source comprises:
operating the thermal energy source so that a line of a cleavage occurs within the patient's skin two to five days following the operating of the thermal energy source, the line of cleavage occurring between the first region and the second region.

10. The method of claim 9, wherein operating the thermal energy source comprises:
operating the energy source in order to form a line of cleavage from two to three cells deep in the stratum corneum of the superficial epidermis and the upper dermis.

11. The method of claim 9, wherein operating the thermal energy source comprises:
operating the thermal energy source such that the tissue in the first region is sloughed tissue.

12. The method of claim 11, further comprising:
removing the sloughed tissue once a new epidermis has been substantially generated in the region of the line of cleavage.

13. The method of claim 9, wherein tissue below the line of cleavage in the second region includes the lower epidermis, the basal membrane, and the DE Junction.

14. The method of claim 12, wherein at least the basal membrane and the DE Junction are regenerated.

15. The method of claim 9, wherein operating the thermal energy source comprises:
operating the thermal energy source such that the line of cleavage forms below areas of solar elastosis and the solar elastosis and deranged fibroblasts are sloughed.

16. The method of claim 1, wherein operating the thermal energy source comprises:
operating the thermal energy source in order to denature dermal collagen in the second region.

17. The method of claim 1, wherein operating the thermal energy source comprises:
operating the thermal energy source such that tissue in the second region undergoes a regenerative process following regeneration of the epidermis.

18. The method of claim 17, wherein operating the thermal energy source comprises:
operating the thermal energy source that a reticular architecture of the dermis is at least partially regenerated by fibroblasts less exposed to the effects of UV radiation.

19. The method of claim 17, wherein operating the thermal energy source comprises:
operating the thermal energy source such that a collagen architecture of the dermis is at least partially regenerated by fibroblasts less exposed to the effects of UV radiation.

20. The method of claim 17, wherein operating the thermal energy source comprises:
operating the thermal energy source such that an elastin architecture of the dermis, is at least partially regenerated by fibroblasts less exposed to the effects of UV radiation.

21. The method of claim 17, wherein operating the thermal energy source comprises:
operating the thermal energy source such that the GAGS of the dermis is at least partially regenerated by fibroblasts less exposed to the effects of UV radiation.

22. The method of claim 1, wherein operating the thermal energy source comprises:
operating the thermal energy source such that a risk of scarring and hypo pigmentation during healing process is substantially eliminated.

23. The method of claim 1, wherein operating the thermal energy source comprises:
operating the thermal energy source such that a progressive improvement in skin changes associated with ageing and photodamage occur over a period of between six and twelve months following the operating of the thermal energy source.

24. The method of claim 1, wherein operating the thermal energy source comprises:
operating an instrument having an electrode connected to a power output device;
creating an electric field in the region of the electrode;
generating a plasma by directing a flow of gas through the electric field, the plasma generated by virtue of the interaction of the electric field with the gas;
directing the plasma onto the tissue for a predetermined period of time; and
controlling power transferred into the plasma from the electric field so as to desiccate at least a portion of the dermis with vapour pockets formed in dermis cells.

25. The method of claim 24, further comprising:
operating the power output device to deliver discrete pulses of heat of millisecond duration.

26. The method of claim 25, wherein operating the power output device comprises:
operating the power output device such that the pulses have a duration between 0.5 to 100 milliseconds.

27. The method of claim 26, wherein operating the power output device comprises:
operating the power output device such that the pulses have a duration between 4.5 to 15.4 milliseconds.

28. The method of claim 24, further comprising:
operating the power output device to deliver between 1 Joule to 4 Joules of energy.

29. A source of thermal energy with a low time constant, for performing the method of claim 1 in therapy.

30. A source of thermal energy with a low time constant for performing the method of claim 1 in regeneration of a reticular architecture of tissue.

31. The use of a source of thermal energy with a low time constant in the preparation of an apparatus for performing the method of claim 1 in regeneration of a reticular architecture of tissue.
